(19) United States
(12) Patent Application Publication
(10) Pub. No.: US 2019/0311804 A1
(43) Pub. Date: Oct. 10, 2019

(54) MANAGING MEDICAL DATA

(71) Applicant: Covidien LP, Mansfield, MA (US)

(72) Inventors: Robert T. Boyer, Longmont, CO (US); William Jordan, Westminster, CO (US)

(21) Appl. No.: 15/948,216

(22) Filed: Apr. 9, 2018

(51) Int. Cl. G16H 40/67; G16H 50/20; G16H 30/20; G06F 17/27

(52) U.S. Cl. CPC G16H 40/67; G06F 17/2705; G16H 30/20; G16H 50/20

(57) ABSTRACT

Methods, systems, and devices for patient monitoring are described. The method may include receiving medical data associated with a measurement by a medical device. The method may also include parsing, from the medical data, a physiological parameter associated with the measurement, a measurement value associated with the physiological parameter, and metadata that indicates how the measurement was performed. The method may further include storing the physiological parameter, the measurement value, and the metadata separately in a table and transmitting, to a display, the measurement value and an indication of the physiological parameter. In some cases, the display may be configured based on the metadata.

### BACKGROUND

The following relates generally to patient monitoring, and more specifically to managing medical data.

In a healthcare facility such as a hospital, physiological parameters of the patient (e.g., heart rate, respiratory rate, blood pressure) may be monitored by one or more medical devices. The medical devices may be battery powered and may wirelessly transmit medical data over a wireless network within the hospital, thereby allowing the patient to move freely through the hospital while being monitored. Clinicians may remotely monitor the patient by accessing the patient medical data at a central nurse station or on any web enabled device connected to the network (e.g., smartphone or tablet).

Medical data being transmitted from networked medical devices is typically formatted using a flat organizational data structure. For example, a piece of data from a medical device may convey the particular type of measurement taken and other details associated with the measurement (e.g., an aortic blood pressure taken with a particular sensor at a particular body location) in the form of a unique name. This flat organizational structure may inhibit the ability to manipulate the medical data once received or to otherwise use the medical data in downstream systems.

### SUMMARY

The described features generally relate to methods, systems, devices, or apparatuses that support managing medical data. A medical data server may receive medical data associated with a measurement by a medical device. The medical data may include a physiological parameter associated with the measurement, a measurement value of the physiological parameter, and metadata that may indicate how the measurement was performed. The medical data server may also separate (e.g., parse), from the medical data, the physiological parameter, the measurement value, and the metadata and store each of the physiological parameter, the measurement value, and the metadata separately in a table.

The ability to parse various parameters from the medical data and store them separately may facilitate the ability to manipulate the medical data (e.g., group the data by conceptual physiological parameter, sort by priority, etc.), dynamically configure displays and other outputs based on the medical data, and to more efficiently input the medical data into downstream systems (e.g., clinical decision support systems).

In some examples, the medical data server may transmit, to a display, the measurement value and an indication of the physiological parameter (e.g., parameter name, alarm threshold range, etc.) and configure the display accordingly. The display may be included in devices such as phones, tablets, computers, or the like.

In some examples, the medical data server may transmit the measurement value and the indication of the physiological parameter to a clinical decision support system based on the metadata. The medical data server may also retrieve additional metadata from a database. In some examples, the medical data server may prioritize a plurality of measurement values associated with the physiological parameters and configure the display based on the prioritization. In other examples, the display may further be configured to indicate the physiological parameter, the measurement value, a unit of measure associated with the measurement value, or the like to the patient, the clinician, or both in a preferred configuration.

A method of patient monitoring is described. The method may include receiving medical data associated with a measurement by a medical device, parsing, from the medical data, a physiological parameter associated with the measurement, a measurement value associated with the physiological parameter, and metadata that indicates how the measurement was performed, storing the physiological parameter, the measurement value, and the metadata separately in a table, and transmitting, to a display, the measurement value and an indication of the physiological parameter, wherein the display is configured based at least in part on the metadata.

Some examples of the method described herein may further include operations, features, means, or instructions for prioritizing a plurality of measurement values associated with the physiological parameter based at least in part on the metadata. In some examples of the method described herein, the prioritizing may be based at least in part on a reliability or a sensitivity of the medical device or of a sensor associated with the medical device.

In some examples of the method described herein, the prioritizing may be based at least in part on a configurable preference based at least in part on the metadata. Some examples of the method described herein may further include operations, features, means, or instructions for selecting one of the plurality of measurement values based at least in part on the prioritizing, wherein the display may be further configured based at least in part on the selected measurement value.

Some examples of the method described herein may further include operations, features, means, or instructions for configuring the display to indicate the physiological parameter, the measurement value, a unit of measure associated with the measurement value, or a combination thereof. Some examples of the method described herein may further include operations, features, means, or instructions for configuring the display to conform to a default display of the medical device based at least in part on the metadata.

Some examples of the method described herein may further include operations, features, means, or instructions for inputting the measurement value and the indication of the physiological parameter into a clinical decision support system based at least in part on the metadata. Some examples of the method described herein may further include operations, features, means, or instructions for parsing, from the medical data, a status associated with the medical device, wherein the status comprises a reset mode, a standby mode, or both.

Some examples of the method described herein may further include operations, features, means, or instructions for parsing, from the medical data, an event associated with the physiological parameter and the metadata, wherein the event comprises an action associated with a patient, an action associated with the medical device, or both. Some examples of the method described herein may further include operations, features, means, or instructions for parsing, from the medical data, an alarm associated with the a physiological condition of a patient, a status associated with the medical device, or both, wherein the alarm comprises a severity of the alarm, an informatory message, or both.

Some examples of the method described herein may further include operations, features, means, or instructions for configuring the display to indicate an alarm threshold range based at least in part on a relationship between the physiological parameter and the alarm. Some examples of the method described herein may further include operations, features, means, or instructions for configuring the display to indicate an alarm threshold range based at least in part on a relationship between the metadata the alarm.

Some examples of the method described herein may further include operations, features, means, or instructions for retrieving, from a database, additional metadata based at least in part on the metadata parsed from the medical data. In some examples of the method described herein, the additional metadata comprises a manufacturer of the medical device, a model number of the medical device, or both.

In some examples of the method described herein, the metadata further indicates an identification of the medical device, or a bodily location of the medical device when the measurement was performed, or both. In some examples of the method described herein, the medical data comprises Health Level-7 (HL7) data.

### BRIEF DESCRIPTION OF THE DRAWINGS

FIG. 1 illustrates an example of a system for wireless communication that supports managing medical data in accordance with aspects of the present disclosure.

FIG. 2 illustrates an example of a system that supports managing medical data in accordance with aspects of the present disclosure.

FIG. 3 illustrates an example of a display configuration that supports managing medical data in accordance with aspects of the present disclosure.

FIG. 4 illustrates an example of a process flow that supports managing medical data in accordance with aspects of the present disclosure.

FIGS. 5 through 7 show block diagrams of a device that supports managing medical data in accordance with aspects of the present disclosure.

FIG. 8 illustrates a block diagram of a system including a medical data server that supports managing medical data in accordance with aspects of the present disclosure.

FIGS. 9 through 13 illustrate methods for managing medical data in accordance with aspects of the present disclosure.

### DETAILED DESCRIPTION

In some patient monitoring systems, a medical device may measure one or more physiological parameters of a patient and communicate medical data associated with the measurements to a central server or some other downstream device or system. In some cases, medical data may be formatted and transmitted using a flat organizational data structure that conveys the particular type of measurement taken and other details associated with the measurement as a single piece of data (e.g., essentially a unique name). However, it may be advantageous to normalize the medical data for subsequent analysis and use in downstream systems. In accordance with aspects of the present disclosure, the central server may separate the medical data into one or more components, store the components separately, and identify and store referential linkages between the different components. As part of this normalization process, the central server may make a distinction between the conceptual physiological data (e.g., a patient's blood pressure) and the sensor or measurement-specific data conveyed by the flat medical data format (e.g., aortic blood pressure taken with a particular sensor). The techniques described herein for processing, storing, and organizing the medical data may increase the efficiency and capabilities associated with leveraging the medical data for use in downstream systems.

The medical device associated with the patient may transmit the medical data to the central server. The central server may then analyze information included in the medical data and parse the information into separate components. For example, the medical data may include a name of the physiological parameter, but the medical data may also include information related to a specific type of the parameter, where the parameter was measured, a value of the parameter, or the like. For example, if the central server receives medical data tagged as aortic blood pressure, the central server may then parse the information embedded within aortic blood pressure data to identify that the medical data includes blood pressure measured in the artery by an arterial catheter and the value of the aortic blood pressure. That is, the central server can distinguish between the conceptual physiological parameter (e.g., blood pressure) and the sensor or measurement-specific metadata associated with that measurement (e.g., the fact that the blood pressure measurement is an aortic blood pressure measurement taken by an arterial catheter). In that case, the central server serves as a mapping tool to identify the different components embedded within the medical data.

In some examples, the physiological parameter may include different types of the same physiological parameter based on the location where the physiological parameter is measured (e.g., information included in metadata). For example, medical devices may measure the blood pressure (e.g., physiological parameter) of the patient. However, the types of blood pressure may include invasive blood pressure, aortic blood pressure, arterial blood pressure, femoral blood pressure, or the like depending on the metadata (e.g., the location where the blood pressure was measured). In that case, the central server may separate each type of blood pressure into categories associated with the physiological parameter, the measurement value, and the metadata (e.g., how the measurement was performed, the location, the type of medical device, etc.).

In some cases, the central server may prioritize the medical data and configure a display (e.g., of a remote device) based on the prioritization. In some examples, the medical data may be prioritized based on a user preference. For example, the central server may receive different types of physiological parameters for the same physiological parameter, and the central server may configure the display to present a single physiological parameter (e.g., based on a prioritization of the different options) requested by the patient or clinician. In other examples, the metadata may be prioritized based on the quality of the medical sensor, a reliability associated with medical device, a sensitivity of the physiological parameter, or a combination thereof.

In some examples, the medical data may be separated into one or more categories. The one or more categories may include the physiological parameter, the metadata, a status associated with the medical device, an event, or an alarm. A relationship may be identified between one or more categories. For example, a relationship may exist between the physiological parameter and the metadata or the physiological parameter and the alarm. The physiological parameter may include a measurement value, and the metadata may include information about how the measurement was performed. In some cases, a user interface display may be configured based on the categories and the relationships. This organizational structure allows the clinician to plug in a clinician decision support algorithm and accommodate the algorithm to corresponding medical data.

The separated categories may be stored and used for further comparisons to allow the clinician to make clinical decisions and perform patient diagnosis. Additionally, the medical data may be separated into individual components so that the information associated with the medical data may not be stored under a single name or in the same place. For example, by separating the medical data into different categories, the central server may store a series of physiological parameters from a patient regardless of the information included in the medical data.

This organizational structure enables the patient, the clinician, or both to search for a given component (e.g., physiological parameter, measurement value, metadata, and other medical data) and analyze the component of choice. In some case, the organizational structure may be used to correlate trends associated with the medical device with outcomes associated with the patient. Further, the central server may include algorithms to analyze the trends, individual components, or relationships to decrease health care costs and allow hospitals and companies to better utilize their resources based on the parsed medical data.

Aspects of the disclosure are initially described in the context of a wireless patient monitoring system. Aspects of the disclosure are further illustrated by and described with reference to apparatus diagrams, system diagrams, and flowcharts that relate to managing medical data.

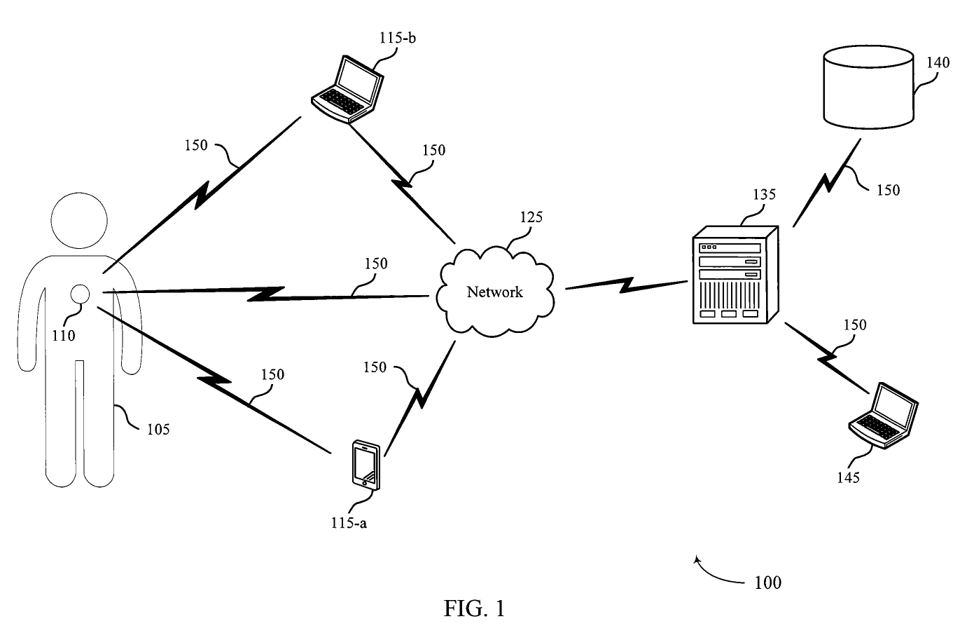

FIG. 1 illustrates an example of a wireless patient monitoring system 100 in accordance with various embodiments of the present disclosure. The wireless patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, or another care facility. The medical device 110 may transmit signals via wireless communications links 150 to computing devices 115 or to a network 125.

The medical device 110 may include one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple local computing devices 115, or any other sensor configured to collect physiological, location, or motion data associated with the patient 105.

The medical device 110 may be coupled with the patient 105 in a variety of ways depending on the data being collected. For example, the medical device 110 may be directly coupled with the patient 105 (e.g., physically connected to the patient's chest, worn around the patient's wrist, attached to the patient's finger, or positioned over the patients nose or mouth). The data collected by the medical device 110 may be wirelessly transmitted to either the computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.).

Computing device 115-a may be a wireless device such as a tablet, cellular phone, personal digital assistant (PDA), a dedicated receiver, or other similar device or a spatially distributed network of devices configured to receive signals from the medical device 110. Computing device 115-b may be a wireless laptop computer, a clinician Workstation on Wheels, or a smart hospital bed configured to receive signals from the medical device 110. The computing devices 115 may be in communication with a central station 135 via network 125.

The medical device 110 may also communicate directly with the central station 135 via the network 125. The central station 135 may be a server or a central nurse station located within the hospital or in a remote location. The central station 135 may be in further communication with one or more remote computing devices 145, thereby allowing a clinician to remotely monitor the patient 105. The central station 135 may also be in communication with various remote databases 140 where the collected patient data may be stored. In some cases, the remote databases 140 include electronic medical records (EMR) applications for storing and sharing patient data.

In accordance with various embodiments, methods and apparatuses are described for managing medical data. When a medical device associated with a patient measures medical data, one or more parameters of the medical data may be measured. For example, a medical device that measures blood pressure may measure different types of blood pressure at one or more locations in the body, such as aortic blood pressure, non-invasive blood pressure, arterial blood pressure, or the like. In that case, a medical data server (e.g., central station 135) may receive medical data associated with the measurement by the medical device and parse the medical data into one or more components or parameters. For example, medical data server may parse the medical data into a conceptual physiological parameter associated with the measurement, a measurement value associated with the physiological parameter, and metadata that indicates how the measurement was performed. The medical data server may also store each component of the parsed medical data.

In some examples, the medical data server may transmit the measurement value and an indication of the physiological parameter to a display (e.g., computing device 115). The display may be configured based on the metadata to indicate the physiological parameter, the measurement value, a unit of measure associated with the measurement value, or the like. In other examples, the medical data server may retrieve, from database 140, additional metadata based on the metadata parsed from the medical data.

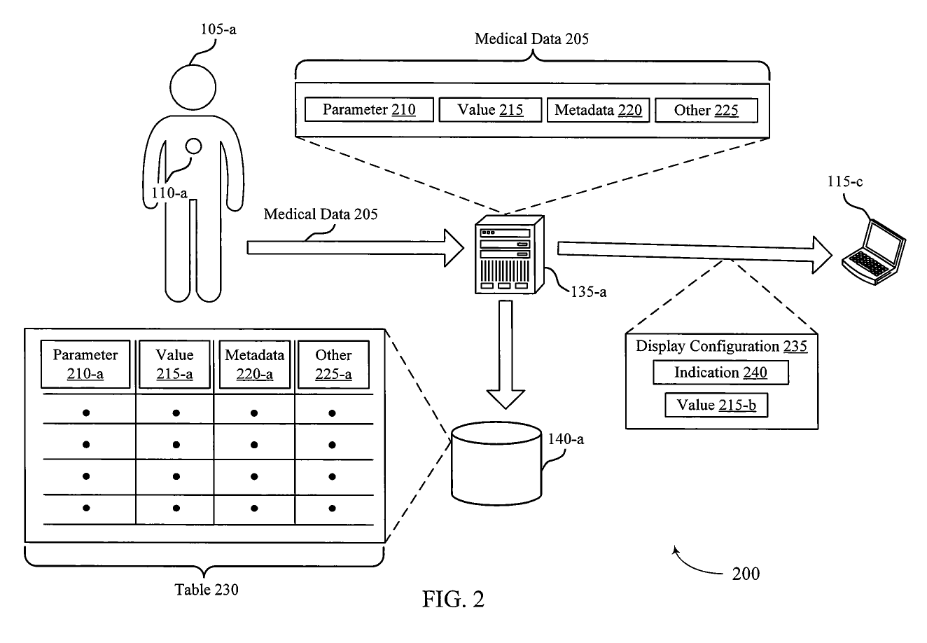

FIG. 2 illustrates an example of a system 200 that supports managing medical data in accordance with aspects of the present disclosure. In some examples, system 200 may implement aspects of wireless patient monitoring system 100 and may include a patient 105-a wearing, carrying, or otherwise coupled with a medical device 110-a. System 200 may be in communication with medical data server 135-a, database 140, and computing device 115-c.

Medical device 110-a may transmit medical data 205 to medical data server 135-a. Medical data server 135-a may be an example of aspects of central station 135. Medical data 205 may include information associated with medical device 110-a. In some cases, medical data 205 may be from one or more sensors associated with medical device 110-a. For example, ECG sensor may include one or more leads where each lead is connected to an electrode. That is, each lead may be placed in different bodily locations. Medical data 205 may include parameter 210 (e.g., physiological parameter), value 215 (e.g., measurement value), and metadata 220. In other examples, medical data may include other medical data 225. Medical data 205 may be an example of Health Level-7 (HL7) data. In some cases, medical data in the form of HL7 data may be parsed and stored.

Medical data server 135-a may normalize (e.g., parse) medical data 205 into different components. Medical data server 135-a may parse from medical data 205-a parameter 210, value 215 of parameter 210, and metadata 220. Medical data server 135-a may further normalize medical data 205 and parse from medical data 205 other medical data 225. For example, other medical data 225 may include device status, events, and alarms.

Parameter 210 may indicate the conceptual physiological parameter measured. For example, parameter 210 may indicate that the physiological parameter being measured is blood pressure, heart rate, blood glucose level, blood oxygen saturation, or the like; however, the parameter 210 is distinct from metadata associated with how the measurement was taken, such as where each physiological parameter is measured or what type of physiological parameter is measured. This distinction between the conceptual physiological parameter being measured (e.g., blood pressure) and the sensor or measurement-specific physiological parameter (e.g., aortic blood pressure taken from a particular location using a particular device) is in contrast to how the medical data is typically formatted and transmitted (e.g., as in the case with HL7 data).

In some cases, parameter 210 may include a unit of measure associated with the physiological parameter. Parameter 210 may be an example of a physiological parameter measured directly from medical device 110-a (e.g., blood pressure) or a computed parameter calculated from one or more physiological parameters measured directly from one or more medical device 110-a.

Metadata 220 may include one or more settings associated with parameter 210. For example, metadata 220 may indicate how the measurement was performed, the bodily location on patient 105-a where the measurement was performed (e.g., location of medical device 110-a, location of a sensor associated with medical device 110-a, etc.), the type of physiological parameter measured, an identification of medical device 110-a, an identification of sensor associated with medical device 110-a, or a combination thereof. For example, metadata 220 may indicate if the blood pressure of patient 105-a was measured at the right forefinger or the femoral artery.

Metadata 220 may also include information associated with medical device 110-a such as a manufacturer name, model number, or both. In some cases, metadata 220 may include an accuracy (e.g., sensitivity) associated with parameter 210 and a quality (e.g., reliability) associated with medical device 110-a. For example, the accuracy may include the standard deviation of the quality of the signal received from medical device 110-a. In some examples, the quality of the sensor associated with medical device 110-a may be cross-referenced against product literature associated with the manufacturer of the sensor.

In some examples, medical data server 135-a may parse a device status from medical data 205. For example, the device status may indicate a status or state of medical device 110-a. That is, the device status may indicate medical device 110-a may be in a standby mode, a reset mode, a recycle mode, or an active mode.

Medical data server 135-a may also parse an event from medical data 205. In some examples, the event may include information associated with patient 105-a (e.g., an action associated with patient 105-a). For example, the event may indicate if patient 105-a takes their medication, at what time patient 105-a takes their medication, and an effect of patient 105-a taking their medication. That is, the event may indicate a change in value 215 of parameter 210 if patient 105-a takes their medication. Alternatively, the event may indicate if patient 105-a misses a dose of their medication and the effect of the missed dosed. For example, the event may indicate a change in value 215 of parameter 210 if patient 105-a misses their medication dose. In some cases, the event may include information associated with medical device 110-a. For example, the event may indicate if medical device 110-a administered the correct or incorrect dose of medication.

In some examples, medical data server 135-a may parse an alarm from medical data 205. For example, the alarm may be associated with a physiological condition of patient 105-a. That is, the alarm may be an example of a physiological alarm. The alarm may indicate a low, medium, or high severity level associated with the physiological condition exceeding an alarm threshold. For example, if the medical data server 135-a parses an alarm that indicates a high severity level, value 215 associated with the measured physiological condition may exceed the alarm threshold. In some cases, the alarm may be associated with a status of medical device 110-a. That is, the alarm may be an example of a technical alarm. For example, the alarm may include an advisory message (e.g., informatory message). In those examples, the alarm may indicate a setting change associated with medical device 110-a.

In some cases, medical data server 135-a may identify a relationship between one or more parsed categories. In some cases, medical data server 135-a may associate metadata 220 with parameter 210. For example, parameter 210 (e.g., blood pressure) may be associated with metadata 220 such as the type of physiological parameter measured (e.g., systolic blood pressure). In some cases, the alarm may be associated with parameter 210. In other examples, the alarm may be associated with metadata 220. For example, a bodily location of medical device 110 (e.g., blood pressure monitor at the right forefinger) may be associated with a corresponding alarm.

Medical data server 135-a may also identify a relationship between the event and parameter 210. For example, medical data server 135-a may associate parameter 210 (e.g., blood pressure) with an event (e.g., inserting an arterial line catheter to measure blood pressure, replacing the arterial line catheter, etc.). In some cases, medical data server 135-a may identify a relationship between the event and parameter 210 for patient diagnosis. For example, in neonatal medicine, a clinician may check the infant for congenital heart defect (CCHD) by placing a sensor to measure the blood oxygen concentration on the foot of the infant and the finger of the infant. The placement of the sensor may be an example of an event, and the blood oxygen concentration measurement, to check the infant for CCHD, may be an example of parameter 210. In this case, the same sensor may be placed in different locations of the body to measure the same parameter 210. If there is a difference between parameter 210 measured at the foot versus parameter 210 measured at the finger, the infant may be susceptible to CCHD. Therefore, the relationship between the event and parameter 210 may aid in patient diagnosis.

Medical data server 135-a may communicate with database 140-a. Database 140-a may be an example of aspects of database 140. Medical data server 135-a may transmit parsed medical data 205 and store medical data 205 in table 230. That is, each parsed component (e.g., parameter 210-a, value 215-a, metadata 220-a, and other medical data 225-a) of medical data 205 may be stored separately in table 230. Medical data server 135-a may also retrieve, from database 140-a, additional metadata 220 based on metadata 220 parsed from medical data 205.

Medical data server 135-a may also store the identified relationships between one or more categories. For example, a parameter 210 may include a set of alarms. The relationship between parameter 210 and the alarm may allow the clinician to configure a multi-parameter alarm system to better treat patient 105-a. In some examples, if parameter 210 exceeds an alarm threshold and the relationship between parameter 210 and the alarm is stored, the medical device 110-a may trigger an alarm.

In some cases, medical data server 135-a may prioritize medical data 205 in table 230. For example, value 215-a may be prioritized based on metadata 220-a. In that case, if the clinician requests to view the highest blood pressure measured via the arterial line catheter, table 230 may sort value 215-a from highest value 215-a to lowest value 215-a. In other examples, medical data server 135-a may prioritize medical data 205 in table 230 based on a reliability or a sensitivity of medical device 110-a or sensor associated with medical device 110-a. For example, if the clinician requests to view the systolic blood pressure of patient 105-a, the table may sort value 215-a from most accurate blood pressure to least accurate blood pressure. In some cases, the medical data server 135-a may include a decision tree where the decision tree may be programmed to sort value 215-a and display parameter 210 based on value 215-a.

Medical data server 135-a may transmit display configuration 235 to computing device 115-c. Computing device 115-c may be an example of aspects of computing device 115. Computing device 115-c may include a display that may be configured by the medical data server 135-a to present display configuration 235. Display configuration 235 may include indication 240 and value 215-b.

Medical data server 135-a may configure the display based on metadata 220. In some case, medical data server 135-a may select value 215-a from table 230 and configure the display based on the selected value 215-a. For example, medical data server may transmit display configuration 235 to display the most accurate blood pressure stored in table 230. If medical data server 135-a may not locate the most accurate blood pressure, the medical data server 135-a may transmit display configuration 235 the next most accurate blood pressure.

For example, in the Intensive Care Unit (ICU), medical data server 135-a may transmit a single value 215-b to display to the clinician. Because medical device 110-a may measure multiple measurements, medical data server 135-a may prioritize or rank which sensor associated with medical device 110-a may include the highest accuracy and display value 215-b with the highest accuracy to the clinician. Medical data server 135-a may normalize medical data 205 to reuse medical data 205 without understanding every parameter 210 included in medical data 205. In some case, medical data server 135-a may be compatible with a clinical decision support algorithm that may be clinician specific. For example, medical data server 135-a may input value 215-b and indication 240 into a clinical decision support system based on metadata 220.

In some cases, indication 240 may include an indication of the event. For example, the clinician may annotate the event to indicate when patient 105-a took their medication, thereby causing a change in parameter 210. In some cases, medical data server 135-a may mark the event on a timeline to allow the clinician to review the patient medical history and transmit the marked event in display configuration 235.

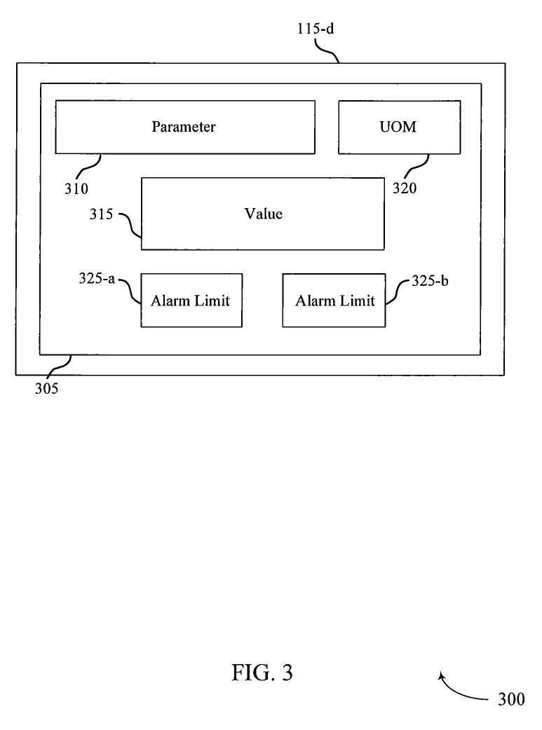

FIG. 3 illustrates an example of a display configuration 300 that supports managing medical data in accordance with aspects of the present disclosure. In some examples, display configuration 300 may implement aspects of wireless patient monitoring system 100. Display configuration 300 may be an example of aspects of system 200 and may include computing device 115-d, display screen 305, parameter 310, value 315, measurement unit 320, alarm limit 325-a, and alarm limit 325-b.

Computing device 115-d may display parameter 310, value 315, measurement unit 320, alarm limit 325-a, and alarm limit 325-b within display screen 305 according to embodiments described herein. Computing device 115-d may be an example of aspects of computing device 115. Display screen 305 may display parameter 310 to indicate the parameter name. Parameter 310 may be an example of aspects of parameter 210, described in reference to FIG. 2. Display screen may also display value 315 and the corresponding measurement unit 320. Value 315 may be an example of the measurement value associated with parameter 310. In some cases, measurement unit 320 may be converted according to user preference.

In some cases, display screen 305 may be configured based on a configurable preference associated with the metadata. In other examples, display screen 305 may be configured based on a default setting (e.g., default display) associated with the medical device measuring parameter 310. For example, a medical device measuring blood pressure may indicate the systolic and diastolic blood pressure within display screen 305. In other examples, parameter 310 and value 315 associated with blood oxygen concentration measured from one medical device may be displayed differently than parameter 310 and value 315 associated with blood oxygen concentration measured from another medical device.

In some examples, the medical data server may configure display screen 305 to display different display styles and display trends based on parameter 310. For example, the display style and display trend may be stored for that specific parameter 310. In that case, the display style may be reused for multiple occurrences of parameter 310. The medical data server may link parameter 310 with a specific configuration to configure display screen 305. For example, a pulse oximeter may transmit values 315 from one or more sensors associated with the pulse oximeter to display screen 305. Independent of the sensor associated with the medical device (e.g., pulse oximeter), display screen 305 may be configured to display parameter 310, value 315, and measurement unit 320 in a common, clear format.

In some cases, display screen 305 may be configured based on the accuracy of value 315 from the medical device. For example, display screen 305 may be configured to display value 315 associated with the respiration of the patient measured from a ventilator rather than value 315 associated with the respiration of the patient computed from a pulse oximeter. That is, the medical data server may configure display screen 305 based on the reliability or sensitivity of the medical device or sensor associated with the medical device.

Display screen 305 may also display an identified relationship between categories, as described in reference to FIG. 2. For example, display screen 305 may indicate alarm limit 325-a and alarm limit 325-b. Alarm limit 325-a may be an example of a low alarm limit, and alarm limit 325-b may be an example of a high alarm limit. In some cases, alarm limit 325-a and alarm limit 325-b may be an example of an alarm threshold range.

In some examples, the medical data server may configure display screen 305 to indicate alarm limit 325-a and alarm limit 325-b based on the relationship between parameter 310 and the alarm. In other examples, the medical data server may configure display screen 305 to indicate alarm limit 325-a and alarm limit 325-b based on the relationship between the metadata associated with parameter 310 and the alarm. For example, alarm limit 325-a and alarm limit 325-b corresponding to blood pressure measured in the femoral artery may be configured differently than alarm limit 325-a and alarm limit 325-b corresponding to blood pressure measured at the forefinger.

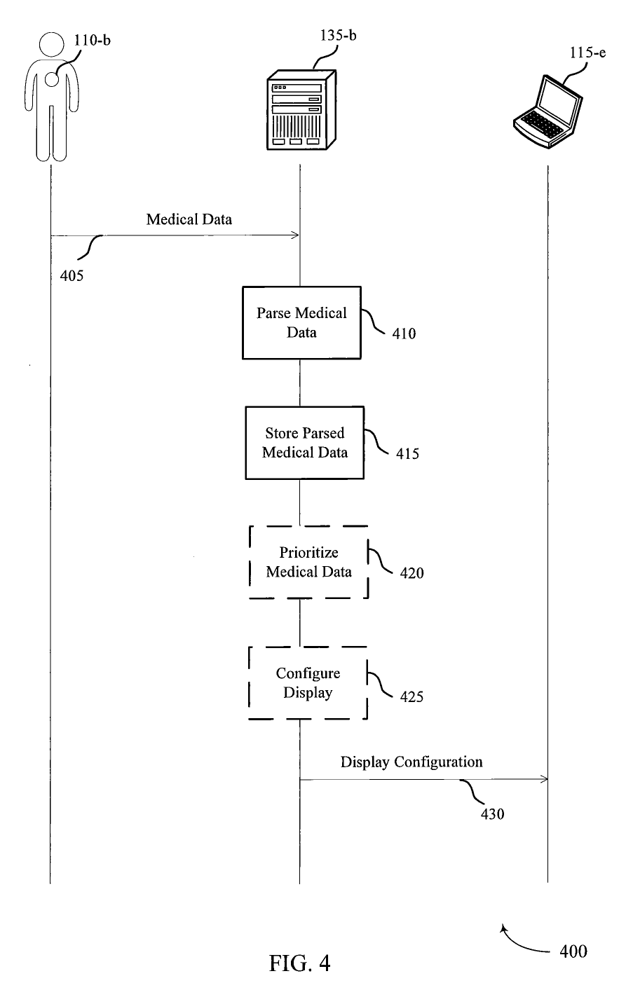

FIG. 4 illustrates an example of a process flow 400 that supports managing medical data in accordance with aspects of the present disclosure. In some examples, process flow 400 may implement aspects of wireless patient monitoring system 100. Process flow 400 may include medical device 110-b, medical data server 135-b, and computing device 115-e which may be respective examples of a medical device 110, central station 135, and computing device 115 as described with reference to FIGS. 1-3. The computing device 115-e may represent a device being used by the patient, the clinician, or both. That is, depending on the particular feature being described below, the signaling being transmitted from the medical data server 135-b to the computing device 115-e may be coming from the patient. Alternative examples of the following may be implemented, where some steps are performed in a different order or not at all. Some steps may additionally include additional features not mentioned above.

In some examples, medical device 110-b may transmit medical data 405, and the medical data server 135-b may receive medical data 405 associated with a measurement by medical device 110-b. Medical data 405 may include HL7 data. Medical data 405 may also include information such as a physiological parameter associated with the measurement, a measurement value associated with the physiological parameter, and metadata that indicates how the measurement was performed. In some cases, the metadata may further indicate an identification of medical device 110-b, or a bodily location of medical device 110-b when the measurement was performed, or both. Medical data server 135-b may also retrieve, from a database, additional metadata based on the metadata parsed from medical data 405. In some examples, the additional metadata may include a manufacturer of medical device 110-b, a model number of medical device 110-b, or both

At block 410, medical data server 135-b may parse (e.g., separate), from medical data 405, the physiological parameter associated with the measurement, the measurement value associated with the physiological parameter, and metadata that indicates how the measurement was performed. In some examples, medical data server 135-b may parse, from medical data 405, a status (e.g., device status) associated with medical device 110-b, wherein the status comprises a reset mode, a standby mode, or both. In other examples, medical data server 135-b may parse, from medical data 405, an event associated with the physiological parameter and the metadata. The event may include an action associated with a patient, an action associated with medical device 110-b, or both. Medical data server 135-b may also parse, from medical data 405, an alarm associated with a physiological condition of a patient, a status associated with the medical device, or both. The alarm may include a severity of the alarm, an informatory message (e.g., advisory message), or both.

At block 415, medical data server 135-b may store the physiological parameter, the measurement value, and the metadata separately in a table. At block 420, medical data server 135-b prioritize a plurality of measurement values associated with the physiological parameter based on the metadata. For example, medical data server 135-b may prioritize the plurality of measurement values associated with the physiological parameter based on a reliability (e.g., quality) or a sensitivity (e.g., accuracy) of medical device 110-b or of a sensor associated with medical device 110-b. In other examples, medical data server 135-b may prioritize the plurality of measurement values associated with the physiological parameter based on a configurable preference based on the metadata. In some cases, medical data server 135-b may select one of the plurality of measurement values based on the prioritizing. For example, a display may be configured based on the selected measurement value.

At block 425, medical data server 135-b may configure the display to indicate the physiological parameter, the measurement value, a unit of measure associated with the measurement value, or a combination thereof. In some examples, medical data server 135-b may configure the display to conform to a default display of medical device 110-b based at least in part on the metadata. In other examples, medical data server 135-b may configure the display to indicate an alarm threshold range (e.g., low alarm limit and high alarm limit) based on a relationship between the physiological parameter and the alarm. Medical data server may configure the display to indicate an alarm threshold range based on a relationship between the metadata and the alarm

In some examples, medical data server 135-b may transmit display configuration 430. For example, medical data server 135-b may transmit, to computing device 115-e, the measurement value and an indication of the physiological parameter. The display may be configured based on the metadata. Transmitting display configuration 430 may be based on the parsed medical data 405 with respect to block 410 and prioritizing medical data 405 with respect to block 420, and the configuration may be conveyed by computing device 115-e in a similar way.

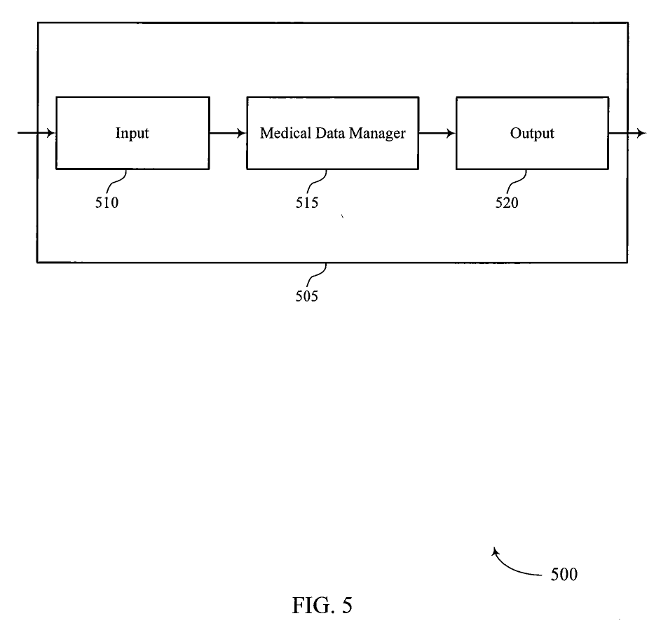

FIG. 5 shows a block diagram 500 of a medical data server 505 that supports managing medical data in accordance with aspects of the present disclosure. Medical data server 505 may be an example of aspects of a medical data server as described herein. Medical data server 505 may include input 510, medical data manager 515, and output 520. Medical data server 505 may also include a processor. Each of these components may be in communication with one another (e.g., via one or more buses).

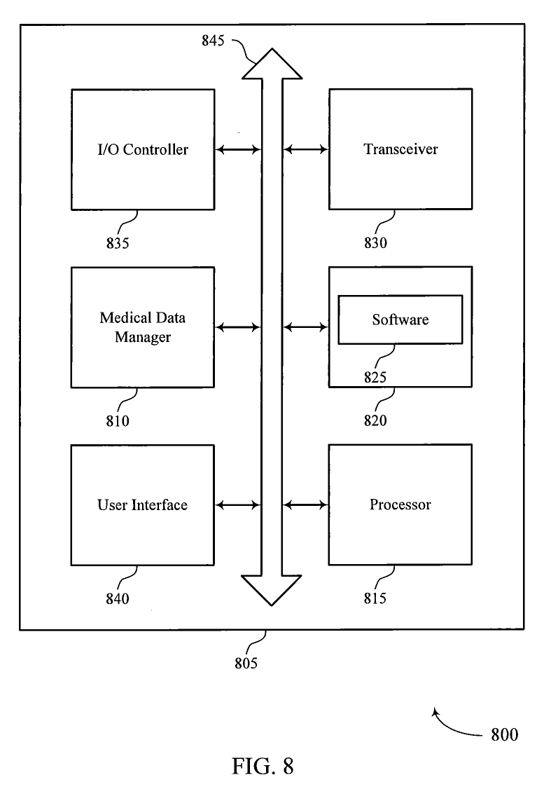

Medical data manager 515 may be an example of aspects of the medical data manager 810 described with reference to FIG. 8.

Medical data manager 515 and/or at least some of its various sub-components may be implemented in hardware, software executed by a processor, firmware, or any combination thereof. If implemented in software executed by a processor, the functions of the medical data manager 515 and/or at least some of its various sub-components may be executed by a general-purpose processor, a digital signal processor (DSP), an application-specific integrated circuit (ASIC), a field-programmable gate array (FPGA) or other programmable logic device, discrete gate or transistor logic, discrete hardware components, or any combination thereof designed to perform the functions described in the present disclosure. The medical data manager 515 and/or at least some of its various sub-components may be physically located at various positions, including being distributed such that portions of functions are implemented at different physical locations by one or more physical devices. In some examples, medical data manager 515 and/or at least some of its various sub-components may be a separate and distinct component in accordance with various aspects of the present disclosure. In other examples, medical data manager 515 and/or at least some of its various sub-components may be combined with one or more other hardware components, including but not limited to an input/output (I/O) component, a transceiver, a network server, another computing device, one or more other components described in the present disclosure, or a combination thereof in accordance with various aspects of the present disclosure.

Medical data manager 515 may receive medical data associated with a measurement by a medical device, parse, from the medical data, a physiological parameter associated with the measurement, a measurement value associated with the physiological parameter, and metadata that indicates how the measurement was performed, store the physiological parameter, the measurement value, and the metadata separately in a table, and transmit, to a display, the measurement value and an indication of the physiological parameter, where the display is configured based on the metadata.

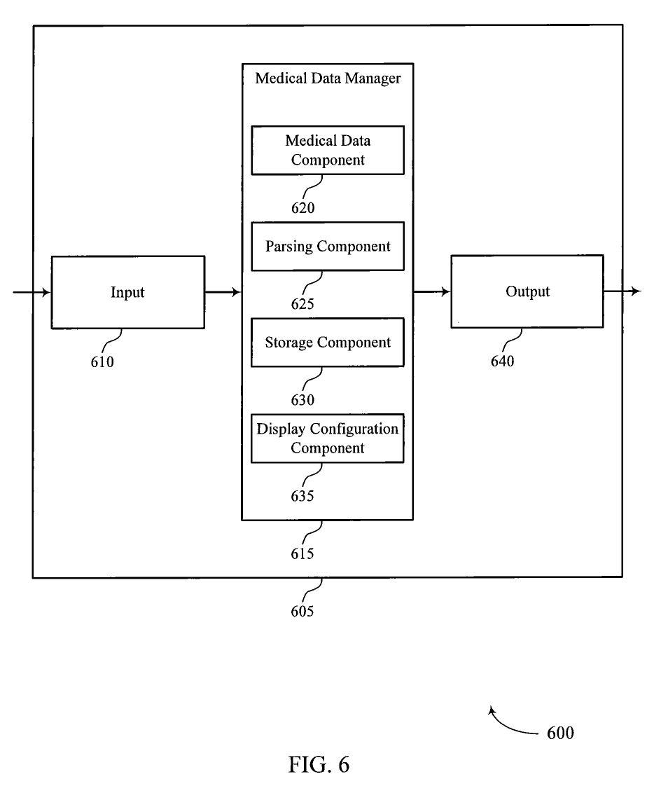

FIG. 6 shows a block diagram 600 of a medical data server 605 that supports managing medical data in accordance with aspects of the present disclosure. Medical data server 605 may be an example of aspects of a medical data server 505 as described with reference to FIG. 5. Medical data server 605 may include input 610, medical data manager 615, and output 640. Medical data server 605 may also include a processor. Each of these components may be in communication with one another (e.g., via one or more buses).

Medical data manager 615 may be an example of aspects of the medical data manager 810 described with reference to FIG. 8.

Medical data manager 615 may also include medical data component 620, parsing component 625, storage component 630, and display configuration component 635.

Medical data component 620 may receive medical data associated with a measurement by a medical device. Medical data component 620 may also input the measurement value and the indication of the physiological parameter into a clinical decision support system based at least in part on the metadata. In some examples, medical data component 620 may retrieve, from a database, additional metadata based at least in part on the metadata parsed from the medical data. In some cases, medical data component 620 may include additional metadata that comprises a manufacturer of the medical device, a model number of the medical device, or both. Medical data component 620 may also include metadata that further indicates an identification of the medical device, or a bodily location of the medical device when the measurement was performed, or both. In some examples, medical data component 620 may include medical data that comprises HL7 data.

Parsing component 625 may parse, from the medical data, a physiological parameter associated with the measurement, a measurement value associated with the physiological parameter, and metadata that indicates how the measurement was performed. Parsing component 625 may also parse, from the medical data, a status associated with the medical device, wherein the status comprises a reset mode, a standby mode, or both. In some examples, parsing component 625 may parse, from the medical data, an event associated with the physiological parameter and the metadata, wherein the event comprises an action associated with a patient, an action associated with the medical device, or both. In other examples, parsing component 625 may parse, from the medical data, an alarm associated with a physiological condition of a patient, a status associated with the medical device, or both, wherein the alarm comprises a severity of the alarm, an informatory message, or both.

Storage component 630 may store the physiological parameter, the measurement value, and the metadata separately in a table.

Display configuration component 635 may transmit, to a display, the measurement value and an indication of the physiological parameter, where the display is configured based on the metadata. Display configuration component 635 may also configure the display to indicate the physiological parameter, the measurement value, a unit of measure associated with the measurement value, or a combination thereof. In some examples, display configuration component 635 may configure the display to conform to a default display of the medical device based at least in part on the metadata. In other examples, display configuration component 635 may configure the display to indicate an alarm threshold range based at least in part on a relationship between the physiological parameter and the alarm. Display configuration component 635 may also configure the display to indicate an alarm threshold range based at least in part on a relationship between the metadata and the alarm.

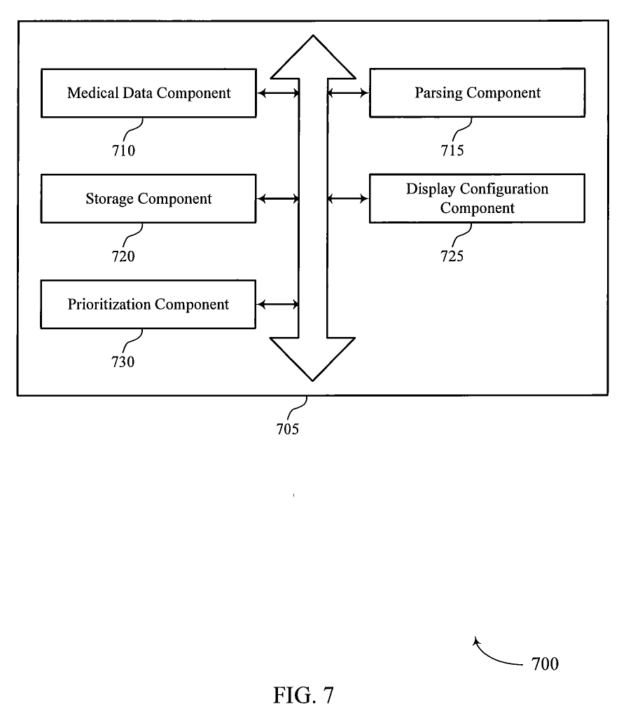

FIG. 7 shows a block diagram 700 of a medical data manager 705 that supports managing medical data in accordance with aspects of the present disclosure. The medical data manager 705 may be an example of aspects of a medical data manager 515, a medical data manager 615, or a medical data manager 810 described with reference to FIGS. 5, 6, and 8. The medical data manager 715 may include medical data component 710, parsing component 715, storage component 720, display configuration component 725, and prioritization component 730. Each of these modules may communicate, directly or indirectly, with one another (e.g., via one or more buses).

Medical data component 710 may receive medical data associated with a measurement by a medical device, input the measurement value and the indication of the physiological parameter into a clinical decision support system based on the metadata, and retrieve, from a database, additional metadata based on the metadata parsed from the medical data. In some cases, the additional metadata includes a manufacturer of the medical device, a model number of the medical device, or both. In some cases, the metadata further indicates an identification of the medical device, or a bodily location of the medical device when the measurement was performed, or both. In some cases, the medical data includes HL7 data.

Parsing component 715 may parse, from the medical data, a physiological parameter associated with the measurement, a measurement value associated with the physiological parameter, and metadata that indicates how the measurement was performed. Parsing component 715 may also parse, from the medical data, a status associated with the medical device, where the status includes a reset mode, a standby mode, or both. In some examples, parsing component 715 may parse, from the medical data, an event associated with the physiological parameter and the metadata, where the event includes an action associated with a patient, an action associated with the medical device, or both. In other examples, parsing component 715 may and parse, from the medical data, an alarm associated with a physiological condition of a patient, a status associated with the medical device, or both, where the alarm includes a severity of the alarm, an informatory message, or both.

Storage component 720 may store the physiological parameter, the measurement value, and the metadata separately in a table.

Display configuration component 725 may transmit, to a display, the measurement value and an indication of the physiological parameter, where the display is configured based on the metadata. Display configuration component 725 may also configure the display to indicate the physiological parameter, the measurement value, a unit of measure associated with the measurement value, or a combination thereof. In some examples, display configuration component 725 may configure the display to conform to a default display of the medical device based on the metadata. In other examples, display configuration component 725 may configure the display to indicate an alarm threshold range based on a relationship between the physiological parameter and the alarm and configure the display to indicate an alarm threshold range based on a relationship between the metadata and the alarm.

Prioritization component 730 may prioritize a set of measurement values associated with the physiological parameter based on the metadata and select one of the set of measurement values based on the prioritizing, where the display is further configured based on the selected measurement value. In some cases, the prioritizing is based on a reliability or a sensitivity of the medical device or of a sensor associated with the medical device. In some cases, the prioritizing is based on a configurable preference based on the metadata.

FIG. 8 shows a diagram of a system 800 including a device 805 that supports managing medical data in accordance with aspects of the present disclosure. Device 805 may be an example of or include the components of medical data server 505 or medical data server 605 as described above, e.g., with reference to FIGS. 5 and 6. Device 805 may include components for bi-directional voice and data communications including components for transmitting and receiving communications, including medical data manager 810, processor 815, memory 820, software 825, transceiver 830, I/O controller 835, and user interface 840. These components may be in electronic communication via one or more buses (e.g., bus 845).

Processor 815 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, processor 815 may be configured to operate a memory array using a memory controller. In other cases, a memory controller may be integrated into processor 815. Processor 815 may be configured to execute computer-readable instructions stored in a memory to perform various functions (e.g., functions or tasks supporting managing medical data).

Memory 820 may include RAM and ROM. The memory 820 may store computer-readable, computer-executable software 825 including instructions that, when executed, cause the processor to perform various functions described herein. In some cases, the memory 820 may contain, among other things, a BIOS which may control basic hardware or software operation such as the interaction with peripheral components or devices.

Software 825 may include code to implement aspects of the present disclosure, including code to support video conferencing and virtual appointments. Software 825 may be stored in a non-transitory computer-readable medium such as system memory or other memory. In some cases, the software 825 may not be directly executable by the processor but may cause a computer (e.g., when compiled and executed) to perform functions described herein.

Transceiver 830 may communicate bi-directionally, via one or more antennas, wired, or wireless links as described above. For example, the transceiver 830 may represent a wireless transceiver and may communicate bi-directionally with another wireless transceiver. The transceiver 830 may also include a modem to modulate the packets and provide the modulated packets to the antennas for transmission, and to demodulate packets received from the antennas.

I/O controller 835 may manage input and output signals for device 805. I/O controller 835 may also manage peripherals not integrated into device 805. In some cases, I/O controller 835 may represent a physical connection or port to an external peripheral. In some cases, I/O controller 835 may utilize an operating system such as iOS®, ANDROID®, MS-DOS®, MS-WINDOWS®, OS/2®, UNIX®, LINUX®, or another known operating system. In other cases, I/O controller 835 may represent or interact with a modem, a keyboard, a mouse, a touchscreen, or a similar device. In some cases, I/O controller 835 may be implemented as part of a processor. In some cases, a user may interact with device 805 via I/O controller 835 or via hardware components controlled by I/O controller 835.

User interface 840 may enable a user to interact with device 805. In some embodiments, the user interface 840 may include an audio device, such as an external speaker system, an external display device such as a display screen, or an input device (e.g., remote control device interfaced with the user interface 840 directly or through the I/O controller module).

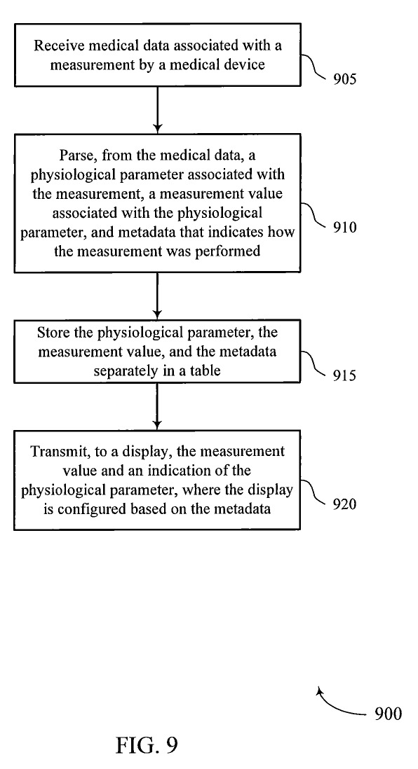

FIG. 9 shows a flowchart illustrating a method 900 for managing medical data in accordance with aspects of the present disclosure. The operations of method 900 may be implemented by a medical data server or its components as described herein. For example, the operations of method 900 may be performed by a medical data manager as described with reference to FIGS. 5 to 8. In some examples, a medical data server may execute a set of codes to control the functional elements of the device to perform the functions described below. Additionally or alternatively, the medical data server may perform aspects of the functions described below using special-purpose hardware.

At 905 the medical data server may receive medical data associated with a measurement by a medical device. The operations of 905 may be performed according to the methods described herein. In certain examples, aspects of the operations of 905 may be performed by a medical data component as described with reference to FIGS. 5 to 8.

At 910 the medical data server may parse, from the medical data, a physiological parameter associated with the measurement, a measurement value associated with the physiological parameter, and metadata that indicates how the measurement was performed. The operations of 910 may be performed according to the methods described herein. In certain examples, aspects of the operations of 910 may be performed by a parsing component as described with reference to FIGS. 5 to 8.

At 915 the medical data server may store the physiological parameter, the measurement value, and the metadata separately in a table. The operations of 915 may be performed according to the methods described herein. In certain examples, aspects of the operations of 915 may be performed by a storage component as described with reference to FIGS. 5 to 8.

At 920 the medical data server may transmit, to a display, the measurement value and an indication of the physiological parameter, where the display is configured based on the metadata. The operations of 920 may be performed according to the methods described herein. In certain examples, aspects of the operations of 920 may be performed by a display configuration component as described with reference to FIGS. 5 to 8.

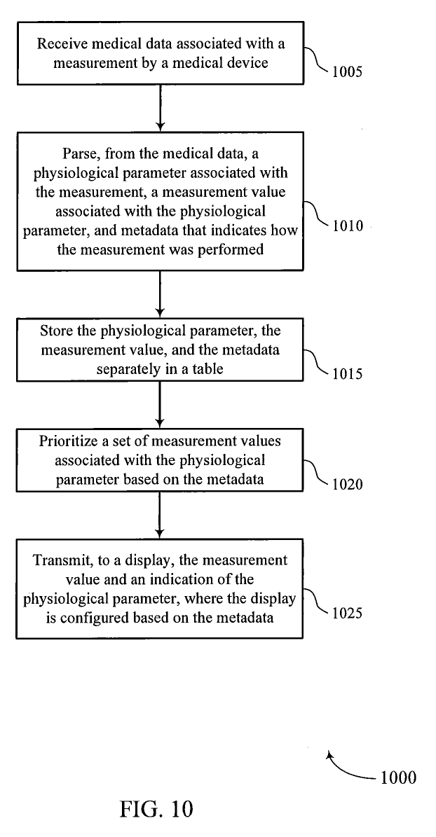

FIG. 10 shows a flowchart illustrating a method 1000 for managing medical data in accordance with aspects of the present disclosure. The operations of method 1000 may be implemented by a medical data server or its components as described herein. For example, the operations of method 1000 may be performed by a medical data manager as described with reference to FIGS. 5 to 8. In some examples, a medical data server may execute a set of codes to control the functional elements of the device to perform the functions described below. Additionally or alternatively, the medical data server may perform aspects of the functions described below using special-purpose hardware.

At 1005 the medical data server may receive medical data associated with a measurement by a medical device. The operations of 1005 may be performed according to the methods described herein. In certain examples, aspects of the operations of 1005 may be performed by a medical data component as described with reference to FIGS. 5 to 8.

At 1010 the medical data server may parse, from the medical data, a physiological parameter associated with the measurement, a measurement value associated with the physiological parameter, and metadata that indicates how the measurement was performed. The operations of 1010 may be performed according to the methods described herein. In certain examples, aspects of the operations of 1010 may be performed by a parsing component as described with reference to FIGS. 5 to 8.

At 1015 the medical data server may store the physiological parameter, the measurement value, and the metadata separately in a table. The operations of 1015 may be performed according to the methods described herein. In certain examples, aspects of the operations of 1015 may be performed by a storage component as described with reference to FIGS. 5 to 8.

At 1020 the medical data server may prioritize a set of measurement values associated with the physiological parameter based on the metadata. The operations of 1020 may be performed according to the methods described herein. In certain examples, aspects of the operations of 1020 may be performed by a prioritization component as described with reference to FIGS. 5 to 8.

At 1025 the medical data server may transmit, to a display, the measurement value and an indication of the physiological parameter, where the display is configured based on the metadata. The operations of 1025 may be performed according to the methods described herein. In certain examples, aspects of the operations of 1025 may be performed by a display configuration component as described with reference to FIGS. 5 to 8.

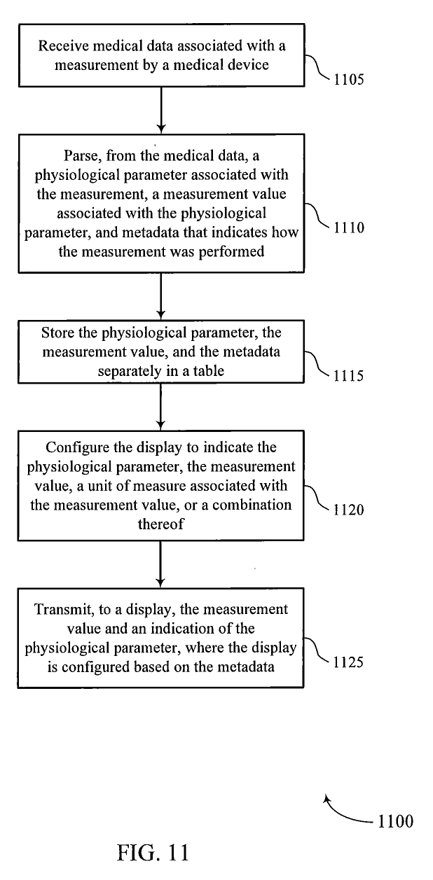

FIG. 11 shows a flowchart illustrating a method 1100 for managing medical data in accordance with aspects of the present disclosure. The operations of method 1100 may be implemented by a medical data server or its components as described herein. For example, the operations of method 1100 may be performed by a medical data manager as described with reference to FIGS. 5 to 8. In some examples, a medical data server may execute a set of codes to control the functional elements of the device to perform the functions described below. Additionally or alternatively, the medical data server may perform aspects of the functions described below using special-purpose hardware.

At 1105 the medical data server may receive medical data associated with a measurement by a medical device. The operations of 1105 may be performed according to the methods described herein. In certain examples, aspects of the operations of 1105 may be performed by a medical data component as described with reference to FIGS. 5 to 8.

At 1110 the medical data server may parse, from the medical data, a physiological parameter associated with the measurement, a measurement value associated with the physiological parameter, and metadata that indicates how the measurement was performed. The operations of 1110 may be performed according to the methods described herein. In certain examples, aspects of the operations of 1110 may be performed by a parsing component as described with reference to FIGS. 5 to 8.

At 1115 the medical data server may store the physiological parameter, the measurement value, and the metadata separately in a table. The operations of 1115 may be performed according to the methods described herein. In certain examples, aspects of the operations of 1115 may be performed by a storage component as described with reference to FIGS. 5 to 8.

At 1120 the medical data server may configure the display to indicate the physiological parameter, the measurement value, a unit of measure associated with the measurement value, or a combination thereof. The operations of 1120 may be performed according to the methods described herein. In certain examples, aspects of the operations of 1120 may be performed by a display configuration component as described with reference to FIGS. 5 to 8.

At 1125 the medical data server may transmit, to a display, the measurement value and an indication of the physiological parameter, where the display is configured based on the metadata. The operations of 1125 may be performed according to the methods described herein. In certain examples, aspects of the operations of 1125 may be performed by a display configuration component as described with reference to FIGS. 5 to 8.

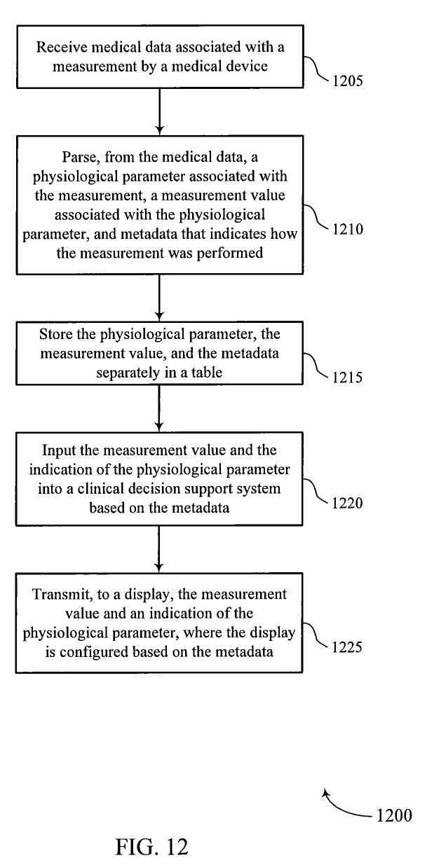

FIG. 12 shows a flowchart illustrating a method 1200 for managing medical data in accordance with aspects of the present disclosure. The operations of method 1200 may be implemented by a medical data server or its components as described herein. For example, the operations of method 1200 may be performed by a medical data manager as described with reference to FIGS. 5 to 8. In some examples, a medical data server may execute a set of codes to control the functional elements of the device to perform the functions described below. Additionally or alternatively, the medical data server may perform aspects of the functions described below using special-purpose hardware.

At 1205 the medical data server may receive medical data associated with a measurement by a medical device. The operations of 1205 may be performed according to the methods described herein. In certain examples, aspects of the operations of 1205 may be performed by a medical data component as described with reference to FIGS. 5 to 8.

At 1210 the medical data server may parse, from the medical data, a physiological parameter associated with the measurement, a measurement value associated with the physiological parameter, and metadata that indicates how the measurement was performed. The operations of 1210 may be performed according to the methods described herein. In certain examples, aspects of the operations of 1210 may be performed by a parsing component as described with reference to FIGS. 5 to 8.

At 1215 the medical data server may store the physiological parameter, the measurement value, and the metadata separately in a table. The operations of 1215 may be performed according to the methods described herein. In certain examples, aspects of the operations of 1215 may be performed by a storage component as described with reference to FIGS. 5 to 8.

At 1220 the medical data server may input the measurement value and the indication of the physiological parameter into a clinical decision support system based on the metadata. The operations of 1220 may be performed according to the methods described herein. In certain examples, aspects of the operations of 1220 may be performed by a medical data component as described with reference to FIGS. 5 to 8.

At 1225 the medical data server may transmit, to a display, the measurement value and an indication of the physiological parameter, where the display is configured based on the metadata. The operations of 1225 may be performed according to the methods described herein. In certain examples, aspects of the operations of 1225 may be performed by a display configuration component as described with reference to FIGS. 5 to 8.

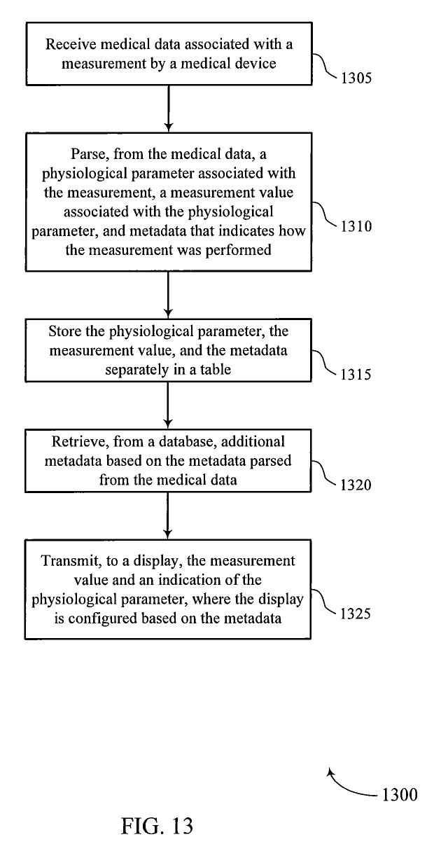

FIG. 13 shows a flowchart illustrating a method 1300 for managing medical data in accordance with aspects of the present disclosure. The operations of method 1300 may be implemented by a medical data server or its components as described herein. For example, the operations of method 1300 may be performed by a medical data manager as described with reference to FIGS. 5 to 8. In some examples, a medical data server may execute a set of codes to control the functional elements of the device to perform the functions described below. Additionally or alternatively, the medical data server may perform aspects of the functions described below using special-purpose hardware.

At 1305 the medical data server may receive medical data associated with a measurement by a medical device. The operations of 1305 may be performed according to the methods described herein. In certain examples, aspects of the operations of 1305 may be performed by a medical data component as described with reference to FIGS. 5 to 8.

At 1310 the medical data server may parse, from the medical data, a physiological parameter associated with the measurement, a measurement value associated with the physiological parameter, and metadata that indicates how the measurement was performed. The operations of 1310 may be performed according to the methods described herein. In certain examples, aspects of the operations of 1310 may be performed by a parsing component as described with reference to FIGS. 5 to 8.

At 1315 the medical data server may store the physiological parameter, the measurement value, and the metadata separately in a table. The operations of 1315 may be performed according to the methods described herein. In certain examples, aspects of the operations of 1315 may be performed by a storage component as described with reference to FIGS. 5 to 8.

At 1320 the medical data server may retrieve, from a database, additional metadata based on the metadata parsed from the medical data. The operations of 1320 may be performed according to the methods described herein. In certain examples, aspects of the operations of 1320 may be performed by a medical data component as described with reference to FIGS. 5 to 8.

At 1325 the medical data server may transmit, to a display, the measurement value and an indication of the physiological parameter, where the display is configured based on the metadata. The operations of 1325 may be performed according to the methods described herein. In certain examples, aspects of the operations of 1325 may be performed by a display configuration component as described with reference to FIGS. 5 to 8.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for patient monitoring, comprising:
receiving medical data associated with a measurement by a medical device;
parsing, from the medical data, a physiological parameter associated with the measurement, a measurement value associated with the physiological parameter, and metadata that indicates how the measurement was performed;
storing the physiological parameter, the measurement value, and the metadata separately in a table; and
transmitting, to a display, the measurement value and an indication of the physiological parameter, wherein the display is configured based at least in part on the metadata.

2. The method of claim 1, further comprising:
prioritizing a plurality of measurement values associated with the physiological parameter based at least in part on the metadata.

3. The method of claim 2, wherein the prioritizing is based at least in part on a reliability or a sensitivity of the medical device or of a sensor associated with the medical device.

4. The method of claim 2, wherein the prioritizing is based at least in part on a configurable preference based at least in part on the metadata.

5. The method of claim 2, further comprising:
selecting one of the plurality of measurement values based at least in part on the prioritizing, wherein the display is further configured based at least in part on the selected measurement value.

6. The method of claim 1, further comprising:
configuring the display to indicate the physiological parameter, the measurement value, a unit of measure associated with the measurement value, or a combination thereof.

7. The method of claim 1, further comprising:
configuring the display to conform to a default display of the medical device based at least in part on the metadata.

8. The method of claim 1, further comprising:
inputting the measurement value and the indication of the physiological parameter into a clinical decision support system based at least in part on the metadata.

9. The method of claim 1, further comprising:
parsing, from the medical data, a status associated with the medical device, wherein the status comprises a reset mode, a standby mode, or both.

10. The method of claim 1, further comprising:
parsing, from the medical data, an event associated with the physiological parameter and the metadata, wherein the event comprises an action associated with a patient, an action associated with the medical device, or both.

11. The method of claim 1, further comprising:
parsing, from the medical data, an alarm associated with a physiological condition of a patient, a status associated with the medical device, or both, wherein the alarm comprises a severity of the alarm, an informatory message, or both.

12. The method of claim 11, further comprising:
configuring the display to indicate an alarm threshold range based at least in part on a relationship between the physiological parameter and the alarm.

13. The method of claim 11, further comprising:
configuring the display to indicate an alarm threshold range based at least in part on a relationship between the metadata and the alarm.

14. The method of claim 1, further comprising:
retrieving, from a database, additional metadata based at least in part on the metadata parsed from the medical data.

15. The method of claim 14, wherein the additional metadata comprises a manufacturer of the medical device, a model number of the medical device, or both.

16. The method of claim 1, wherein the metadata further indicates an identification of the medical device, or a bodily location of the medical device when the measurement was performed, or both.

17. The method of claim 1, wherein the medical data comprises Health Level-7 (HL7) data.

18. An apparatus for patient monitoring, comprising:
a processor;
memory in electronic communication with the processor; and
instructions stored in the memory and executable by the processor to cause the apparatus to:
receive medical data associated with a measurement by a medical device;
parse, from the medical data, a physiological parameter associated with the measurement, a measurement value associated with the physiological parameter, and metadata that indicates how the measurement was performed;
store the physiological parameter, the measurement value, and the metadata separately in a table; and
transmit, to a display, the measurement value and an indication of the physiological parameter, wherein the display is configured based at least in part on the metadata.

19. The apparatus of claim 18, wherein the instructions are further executable by the processor to cause the apparatus to:
prioritize a plurality of measurement values associated with the physiological parameter based at least in part on the metadata.

20. A non-transitory computer readable medium storing code for patient monitoring, the code comprising instructions executable by a processor to:
receive medical data associated with a measurement by a medical device;
parse, from the medical data, a physiological parameter associated with the measurement, a measurement value associated with the physiological parameter, and metadata that indicates how the measurement was performed;
store the physiological parameter, the measurement value, and the metadata separately in a table; and
transmit, to a display, the measurement value and an indication of the physiological parameter, wherein the display is configured based at least in part on the metadata.
